(19) United States
(12) Patent Application Publication
(10) Pub. No.: US 2005/0277831 A1
(43) Pub. Date: Dec. 15, 2005

(54) Method for determining regions of a body part that are undersupplied owing to a vessel closure, and a tomography

(76) Inventor: Lutz Guendel, Erlangen (DE)

Correspondence Address: HARNESS, DICKEY & PIERCE, P.L.C., P.O.BOX 8910, RESTON, VA, 20195, US

(21) Appl. No.: 11/147,187

(22) Filed: Jun. 8, 2005

(30) Foreign Application Priority Data
Jun. 9, 2004 (DE) 10 2004 028 123.8

(51) Int. Cl. A61B 5/05

(52) U.S. Cl. 600/425

(57) ABSTRACT

A method is for determining regions of a body part that are undersupplied owing to a vessel closure, in which a three-dimensional image data record of the body part is compiled with the aid of a tomograph. Vessels of the body part are segmented from the image data record, at least one vessel closure is localized in the segmented vessels, and the region of the body part that is undersupplied owing to the localized vessel closure is determined in the image data record. Further, a residual opening cross section is determined at the localized vessel closure as a ratio of the total cross section of the vessel, and the ratio or a value derived therefrom is visualized. The method permits a more accurate statement on the effects of a vessel closure.

The present application hereby claims priority under 35 U.S.C. §119 on German patent application number DE 10 2004 028 123.8 filed Jun. 9, 2004, the entire contents of which is hereby incorporated herein by reference.

### FIELD

The present invention generally relates to a method for determining regions of a body part that are undersupplied owing to a vessel closure. For example, it relates to one in which a three-dimensional image data record of the body part is compiled with the aid of a tomograph, vessels of the body part are segmented from the image data record, at least one vessel closure is localized in the segmented vessels, and the region of the body part that is undersupplied owing to the localized vessel closure is determined in the image data record. The invention also generally relates to a tomograph for carrying out the method.

### BACKGROUND

One application of a method may be the determination of the regions of the lung that are undersupplied owing to an embolism by using an imaging tomographic technique. Embolisms can obstruct pulmonary arteries either partially or completely. Owing to the reduced blood flow in distally located pulmonary arteries, regions of the lung are supplied with less blood, or even no further blood and can therefore no longer contribute to the exchange of gas.

Computed tomography (CT) pictures of the lung are nowadays taken in clinical practice in conjunction with contrast agent injection in order to determine these undersupplied pulmonary regions. The axial two-dimensional images obtained therefrom are displayed on a display device and searched by the user for the obstructions.

If the radiologist finds a partial or complete vessel closure, he can determine the pulmonary regions affected thereby on the basis of his anatomical knowledge. However, this is performed only very imprecisely, in particular in cases when the closures are located in very small vessels which can be displayed very well with the aid of modern multirow tomographs. However, an accurate quantitative determination of the resonant pulmonary regions is of the greatest importance for the subsequent diagnosis.

For this reason, current developments relate to an improved determination of the pulmonary regions that are undersupplied owing to vessel closure. For this purpose, the three-dimensional image data record, compiled by the CT pictures, of the lung is completely segmented in order to obtain a three-dimensional representation of the artery tree. If a closure is detected in this segmented artery tree either manually by the user or by way of an automatic method (Computer Aided Detection), the relevant distally displaced partial load on the artery tree is determined. The relevant pulmonary region is subsequently delimited and brought into relationship with the total pulmonary volume. The value thereby obtained then reflects the limitation of the pulmonary functionality much better than as the only quantitative analysis in the axial two-dimensional images.

### SUMMARY

An object of an embodiment of the present invention includes specifying a method for determining regions of a body part that are unsupplied owing to a vessel closure, in particular regions of the lung, and/or a tomograph for carrying out the method. As such, it may be possible to make a further improved statement on the effects of the vessel closure.

An object may be achieved by way of a method in accordance with at least one embodiment. Advantageous refinements of the method can be gathered from the following description and the example embodiments.

In a method of at least one embodiment for determining regions of a body part that are undersupplied owing to a vessel closure, a three-dimensional image data record of the body part is compiled by using imaging tomography. Vessels of the body part are segmented from the image data set, a vessel closure is localized in segmented vessels, and the region of the body part that is undersupplied owing to the localized vessel closure is determined in the image data set. In the method of at least one embodiment, a residual opening cross section may be determined at the localized vessel closure as a ratio to the total cross section of the vessel, and the ratio or a value derived therefrom may be visualized.

The user can make substantially more precise statements on the effects of the vessel closure by determining the residual opening cross section at the vessel closure as the ratio of the total cross section of the vessel, and by visualizing this ratio or a value derived therefrom. Thus, specifically in the case of the examination of lungs, the residual pulmonary functionality can be determined substantially more accurately than with the technique described at the beginning, in which the type of vessel closure is not taken into account. It is frequently a question not of a complete interruption of the blood flow, but only of a restriction with reduced blood flow such that the pulmonary region affected or undersupplied can still contribute in a restricted way to the gas exchange.

The percentage calculated with the aid of the technique explained at the beginning certainly thereby constitutes a measure of the relevant pulmonary proportion, but not a measure of the residual pulmonary functionality. This state of affairs is taken into account by quantitative determination, undertaken above, of the residual cross section at the constriction of the vessel closure in relation to the total cross section of the vessel at this point.

In the event of a lack of more accurate knowledge on the capacity for gas exchange in relation to the residual throughflow cross section, a linear relationship may be adopted. In the event of more accurate knowledge, a specific pulmonary model can also be used via which it is then possible to use the ratio of the residual opening cross section to the total cross section of the vessel at the point of the closure to derive a value for the residual capacity for gas exchange, and display it.

The residual pulmonary functionality or the specific ratio can be visualized in various ways. Thus, the determined value can be visualized in a three-dimensional display of the vessel tree as it was obtained by the preceding segmentation, in an overall display of the 3D image data record or in a display of the lung segmented from the 3D image date record, or by color coding of the undersupplied region.

The value of the pulmonary functionality, obtained in the method of at least one embodiment, of the undersupplied region may also be included in the calculation of the overall functionality of the lung. This leads, particularly in the case of partial closures of smaller arteries, or in the case of a number of closures in different pulmonary regions, to a much more precise statement on the pulmonary functionality.

The tomographic imaging may be carried out with the aid of a tomography, for example a multirow tomograph. For the purpose of increasing the contrast, the image recording may be performed in this case, for example, with injection of a contrast agent. The raw data obtained in this case are used to reconstruct image data in a known way for a multiplicity of axial 2D tomograms from which a three-dimensional image data record of the examined body part results on the basis of the known slice spacing.

After the segmentation of the vessels from this image data record and the localization of one or more vessel closures, the residual vessel cross section is then determined as a ratio to the total cross section of the vessel at the point of the respective vessel closure. This can be performed in the case of a CT picture by segmenting and measuring the bolus forming the obstruction via the density values or CT numbers, on the one hand, and by measuring the total vessel cross section, on the other hand, the latter preferably likewise after a segmentation of the vessel.

All the segmentation steps can be performed in the case of the present method in accordance with a stipulation of the corresponding limiting values for the segmentation. The localization of the vessel closures can also be carried out automatically via an image processing program that recognizes constrictions or closures in the segmented course of the vessel. The measurement of the bolus and of the entire vessel cross section at this point can be carried out automatically with the aid of a suitable image processing program for example, semiautomatically, and/or can be undertaken manually by the user at the display device.

In addition to the determination of pulmonary regions undersupplied owing to an embolism, at least one embodiment of the method can also, of course, be used to determine regions of other body parts that are undersupplied owing to vessel closure. In these cases, it may then not be the pulmonary functionality, but the measure of undersupplying of the relevant region that is determined on the basis of the constricted or closed point of the vessel.

Furthermore, instead of the imaging technique of computed tomography, it is also possible to use other imaging tomographic recording methods with the aid of which it is possible to compile three-dimensional image data records of the body part. All that is important here is to be able to segment the vessels from this image data set. Non-limiting examples of such tomographic imaging techniques are magnetic resonance tomography or ultrasound tomography.

An object relating to the tomographs may be achieved by way of a tomograph that is set up to carry out the method of at least one embodiment explained above. For this purpose, the tomograph may include an image computer with an image processing program for automatically carrying out the steps of segmentation, localization and measurement in the method according to at least one embodiment of the invention.

### BRIEF DESCRIPTION OF THE DRAWINGS

Embodiments of the invention is explained briefly once more below with the aid of the drawings, in which:

FIG. 1 shows a schematic of a tomograph for carrying out at least one embodiment of the present method; and

FIG. 2 shows a schematic of an exemplary sequence at least one embodiment of the present method.

### DETAILED DESCRIPTION OF THE EXAMPLE EMBODIMENTS

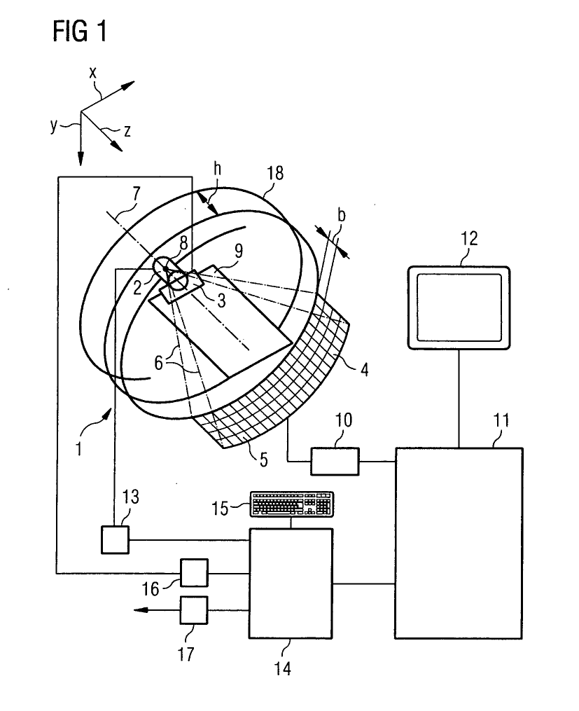

A third-generation tomograph 1 is illustrated schematically in FIG. 1. Its measuring arrangement has an X-ray emitter 2 with an insertion device 3 in front thereof and near the source, and an X-ray detector 5 designed as a multirow or planar array of a number of rows and columns of detector elements 4. For the sake of clarity, only four rows of detector elements 4 are illustrated in the illustration of FIG. 1. However, the X-ray detector can have further rows of detector elements 4, including a different width b. The X-ray detector 5 can be designed as a solid state matrix detector system, in particular as a flat image detector and/or as a detector that comprises a scintillator layer and an assigned electronic photoreceiver matrix.

The X-ray emitter 2 with the insertion device 3, on the one hand, and the X-ray detector 5 with its radiation diaphragm (not illustrated), on the other hand, are fitted on a rotary frame situated opposite one another in such a way that a pyramidal X-ray beam that emanates from the X-ray emitter 2 during operation of the tomograph 1 and is inserted by the insertion device 3, and whose edge rays are denoted in FIG. 1 by the reference numeral 6, strikes the X-ray detector 5. The rotary frame can be set rotating about a system axis 7 via a drive device (not illustrated). The system axis 7 runs parallel to the z-axis of a three-dimensional rectangular coordinate system illustrated in FIG. 1. The columns of the X-ray detector 5 likewise run in the direction of the z-axis, while the rows, whose width b is measured in the direction of the z-axis and is, for example, 1 mm, run transverse to the system axis 7 or the z-axis.

In order to be able to bring the examination object, the patient, into the beam path of the X-ray beam, a bearing device 9 is provided that can be displaced parallel to the system axis 7, that is to say in the direction of the z-axis. The displacement is performed in such a way that there is synchronization between the rotary movement of the rotary frame and the translatory movement of the bearing device 9, it being possible to set the ratio of speed of translation to rotation speed by prescribing a desired value for the feed h of the bearing device 9 per revolution of the rotary frame.

An object volume, which includes the lung in the present case, of a patient situated on the bearing device 9 can be examined by way of volumetric scanning by operating this tomograph 1. In the case of spiral scanning, many projections can be recorded from various projection directions accompanied by rotation of the rotary frame and simultaneous translation of the bearing device 9 and revolution of the rotary frame. In the case of spiral scanning, the focus 8 of the X-ray emitter 2 moves relative to the bearing device 9 on a spiral track 18. Sequence scanning is also possible as an alternative to this spiral scanning.

The measured data, which are read out in parallel during spiral scanning from the detector elements 4 of each active row of the detector system 5 and correspond to the individual projections, are subjected to analog-to-digital conversion in a data conditioning unit 10, serialized and transmitted as raw data to an image computer 11 that displays the result of image reconstruction on the display unit 12, for example a video monitor. Also performed in this image computer is the compilation of a three-dimensional image data record from the reconstructed tomograms, and the segmentation of the vessel tree, included in the 3D image data record, of the examined body part, the lung in the present example. The segmented vessel tree can then be displayed as a three-dimensional display on the display unit 12.

The X-ray unit 2, for example an X-ray tube, is supplied with the necessary voltages and currents by a generator unit 13. In order to be able to set these to the values respectively required, the generator unit 13 is assigned a control unit 14 with a keyboard 15 that allows the appropriate settings. The remaining operation and control of the tomograph 1 is also performed by the control unit 14 and the keyboard 15.

It is possible, inter alia, to set the number of the active rows of detector elements 4, and thus the position of the insertion device 3 and of the optional radiation diaphragm, close to the detector, for which purpose the control unit is connected to adjusting units 16 or 17 assigned to the insertion device 3 and the optional radiation diaphragm close to the detector. Furthermore, it is possible to set the rotation period required by the rotary frame for a complete revolution.

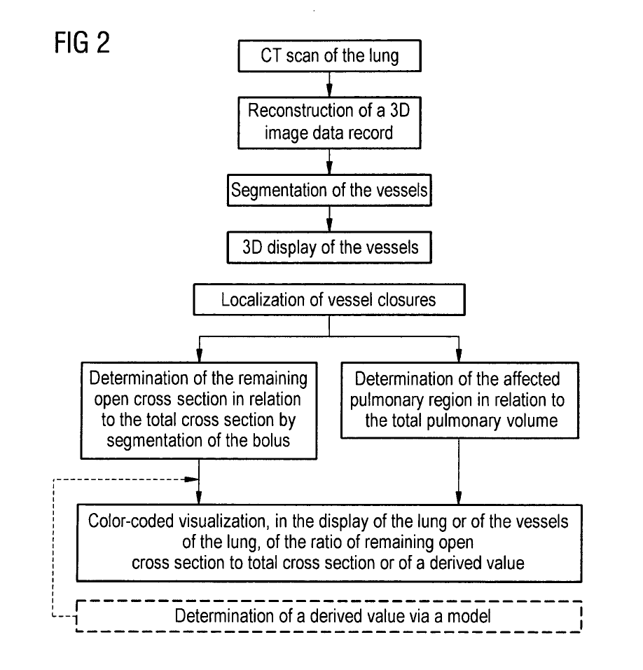

FIG. 2 shows for the purpose of explanation an example of the sequence when carrying out the present method. In this exemplary embodiment, volumetric scanning of an object volume including the patient's lung is carried out, to the accompaniment of the injection of a contrast agent, by the tomograph 1 with the aid of spinal scanning. A three-dimensional image data record of the object volume is reconstructed in the imaging computer from the raw data obtained from the measurement. A 3D image data record is segmented on the basis of the CT values characterizing the contrast agent, in order to obtain the course of the vessels of the arteries of the lung through which contrast agent flows.

Subsequently, the artery tree obtained by segmentation is illustrated in three dimensions for the user at the monitor in a suitable way. The user can then either manually search for vessel closures in the display and mark them interactively, or start a program for automatically localizing these vessel closures.

Subsequently, a segmentation of the plaque plug forming the obstruction is performed with the aid of its CT numbers, which differ from the surrounding tissue and from the contrast agent. The open artery cross section is obtained in relation to the total cross section from a measurement of this segmented plug or bolus and the total cross section of the artery at the point of obstruction. Furthermore, the pulmonary region affected by the respective vessel closure is determined and put into relation with the total pulmonary volume.

Finally, the two lungs and the pulmonary lobes are segmented from the 3D image date record and displayed on the display device. The pulmonary functionality, determined from the ratio of the residual opening cross sections, in the pulmonary region undersupplied owing to the vessel closure is visualized in this case in the display of the lungs and/or pulmonary lobes in this region in a color-coded fashion. In this way, the user firstly immediately detects the relevant pulmonary regions and secondly also detects by the color of these regions the degree of pulmonary functionality remaining owing to the vessel closure.

Any of the aforementioned methods may be embodied in the form of a system or device, including, but not limited to, any of the structure for performing the methodology illustrated in the drawings.

Further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, such as floppy disks (trademark), cassette tapes, and removable hard disks; media with a built-in rewriteable non-volatile memory, such as memory cards; and media with a built-in ROM, such as ROM cassettes.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method, in which a three-dimensional image data record of a body part is compiled with the aid of a tomograph, the method comprising:
segmenting vessels of the body part from the image data record;
localizing at least one vessel closure in the segmented vessels;
determining a region of the body part that is undersupplied owing to the localized vessel closure; and
determining, at the localized vessel closure, a residual opening cross section as the ratio of the total cross section of the vessel, and visualizing at least one of the ratio and a value derived therefrom.

2. The method as claimed in claim 1, wherein, on the basis of a supply model, a value is derived from the ratio that constitutes a measure of a residual supply of the undersupplied region.

3. The method as claimed in claim 1, wherein the vessels segmented from the image data record are displayed in three dimensions on a display device.

4. The method as claimed in claim 1, wherein the at least one of the ratio and the derived value is graphically visualized on a display device in a display of at least one of the segmented vessels, the image data record and elements segmented therefrom.

5. The method as claimed in claim 4, wherein the at least one of the ratio and the derived value is visualized in the display by color coding of the region undersupplied owing to the vessel closure.

6. The method as claimed in claim 1, wherein a total volume of the body part, and a partial volume thereof that is stressed owing to the undersupplied region are determined from the image data record, and wherein the partial volume is brought into relation with the total volume.

7. The method as claimed in claim 1, wherein the method is for determining limitation of pulmonary functionality in the event of closure of pulmonary arteries.

8. Tomograph, comprising an image computer with an image processing program for automatically carrying out the method as claimed in claim 1.

9. The method of claim 1, wherein the determining of a residual opening cross section is achieved by segmentation and measurement of a bolus forming an obstruction via at least one of density values and CT numbers and measurement of the total vessel cross section.

10. The method as claimed in claim 2, wherein the vessels segmented from the image data record are displayed in three dimensions on a display device.

11. The method as claimed in claim 2, wherein the at least one of the ratio and the derived value is graphically visualized on a display device in a display of at least one of the segmented vessels, the image data record and elements segmented therefrom.

12. The method as claimed in claim 3, wherein the at least one of the ratio and the derived value is graphically visualized on a display device in a display of at least one of the segmented vessels, the image data record and elements segmented therefrom.

13. The method as claimed in claim 10, wherein the at least one of the ratio and the derived value is visualized in the display by color coding of the region undersupplied owing to the vessel closure.

14. The method as claimed in claim 2, wherein a total volume of the body part, and a partial volume thereof that is stressed owing to the undersupplied region are determined from the image data record, and wherein the partial volume is brought into relation with the total volume.

15. The method as claimed in claim 3, wherein a total volume of the body part, and a partial volume thereof that is stressed owing to the undersupplied region are determined from the image data record, and wherein the partial volume is brought into relation with the total volume.

16. A method for determining limitation of pulmonary functionality in the event of closure of pulmonary arteries, comprising the method of claim 1.

17. A computer program including program modules for, when executed on a computer device, performing the method as claimed in claim 1.

18. A computer readable medium, comprising the computer program of claim 17.

19. An apparatus, in which a three-dimensional image data record of a body part is compiled with the aid of a tomograph, the apparatus comprising:
means for segmenting vessels of the body part from the image data record;
means for localizing at least one vessel closure in the segmented vessels;
means for determining a region of the body part that is undersupplied owing to the localized vessel closure; and
means for determining, at the localized vessel closure, a residual opening cross section as the ratio of the total cross section of the vessel, and visualizing at least one of the ratio and a value derived therefrom.

20. The apparatus of claim 19, wherein the means for determining is for determining a residual opening cross section by segmentation and measurement of a bolus forming an obstruction via at least one of density values and CT numbers and measurement of the total vessel cross section.
